(19) United States
(12) Patent Application Publication
(10) Pub. No.: US 2009/0053343 A1
(43) Pub. Date: Feb. 26, 2009

(54) Kits and Methods for Assessing Leptin-Mediated Lipid Metabolism

(76) Inventor: Robert P. Ricciardi, Kennet Square, PA (US)

Correspondence Address: BRANDY C HILL, P.O. BOX 951121, LAKE MARY, FL, 32795, US

(21) Appl. No.: 12/259,528

(22) Filed: Oct. 28, 2008

Related U.S. Application Data
(63) Continuation-in-part of application No. 10/480,683, filed on May 6, 2005.
(60) Provisional application No. 60/298,136, filed on Jun. 14, 2001.

(51) Int. Cl. C12Q 1/68

(52) U.S. Cl. 425/6

(57) ABSTRACT

The invention relates to kits and methods for assessing susceptibility of a human to abnormal lipid metabolism and disorders associated therewith, such as obesity and diabetes. The methods involve assessing occurrence in the human's genome of one or more polymorphisms (e.g., single nucleotide polymorphisms) that occur in one or more genes associated with leptin-mediated lipid metabolism and that are associated with a disorder in humans. Preferred assessment and scoring methods are disclosed, as are kits for performing the methods.

### CROSS-REFERENCE TO RELATED APPLICATIONS

This application is a Continuation-in-Part of U.S. patent application Ser. No. 10/480,683 filed May 6, 2005, which is the national stage application of PCT/US02/19018 filed Jun. 14, 2002, now pending and expressly incorporated herein in its entirety, and is entitled to priority pursuant to 35 USC § 120; 35 USC § 365(c), and 37 CFR § 1.78. Priority is further claimed under 35 USC §119(e) to U.S. Provisional Application Ser. No. 60/298,136 filed Jun. 14, 2001, also expressly incorporated herein in its entirety.

### STATEMENT REGARDING FEDERALLY-SPONSORED RESEARCH OR DEVELOPMENT

Not applicable.

### FIELD OF THE INVENTION

The disclosure relates generally to the field of physiological and nutritional assessment of individuals. The disclosure further relates to methods assessing the susceptibility of a human to abnormal lipid metabolism by assessing occurrence in the human's genome of certain polymorphisms: one of which is in the LEP gene and three of which are in the LEPR gene.

### OBJECTS OF THE INVENTION

It is an object of the invention to provide a method for assessing the susceptibility of a human to abnormal lipid metabolism by assessing occurrence in the human's genome of certain polymorphisms.

It is a further object of the invention to provide advisement to individuals who exhibit susceptibility to abnormal lipid metabolism to employ an intervention which may include advising or employing consumption or use of a nutritional supplement or composition, advising or employing a nutritional supplement or composition, or advising or undergoing heightened medical monitoring.

The aforementioned objects of the invention are not intended as limiting on the scope of the invention disclosed herein. Likewise, other and further objects of the invention may be apparent or readily concluded from the detailed description of the invention which follows.

### BEST MODE OF PRACTICING THE INVENTION

The objects (and appurtenant benefits) of the invention are set forth above. By practicing the methods disclosed herein, one skilled in the art can readily predict the predisposition of an individual to abnormal lipid metabolism. This is an important benefit for the reason that individuals who exhibit predisposition to abnormal lipid metabolism may be at further and greater risk for serious disease states such as Gaucher's disease, Wolman's disease, Cerebrotendinous xanthomatosis, Tay-Sachs disease, Neimann-Pick disease, Fabry's disease, and fatty acid oxidation disorders such as medium chain acyl-CoA dehydrogenase (MCAD). Accordingly, the best mode of carrying out the invention is as follows:
a) obtaining a biological sample from the individual;
b) assessing the individual's genome for presence or absence of the following polymorphisms:
   1. a polymorphism manifested as a change from a thymine to a cytosine at nucleotide residue +70 (in the first exon) of the leptin receptor (LEPR) gene;
   2. a polymorphism manifested as a change from an Asp (A) to an Asp (G) at amino acid position 96 (in the fourth exon) of the leptin receptor (LEPR) gene;
   3. a polymorphism manifested as a change from a Ser (T) to a Ser (C) at amino acid position 343 (in the ninth exon) of the leptin receptor (LEPR) gene; and
   4. a polymorphism manifested as a change from a guanine residue to an adenine residue at nucleotide residue 2548 of the leptin (LEP) gene;
c) advising an individual whose genome comprises one or more of the aforementioned polymorphisms he or she exhibits a predisposition for abnormal lipid metabolism;
d) advising an individual whose genome comprises one or more of the aforementioned polymorphisms to employ an intervention.

By practicing the inventor's methods, an individual who exhibits susceptibility to abnormal lipid metabolism can take proactive steps to protect his or her health in an effort to minimize, delay or prevent potential negative health events commonly associated with abnormal lipid metabolism.

### BACKGROUND OF THE INVENTION

Lipid metabolism, glucose metabolism, energy expenditure, and appetite regulation are factors that contribute to maintenance of healthy body weight. Various disorders relate to one or more of these factors. Diabetes is one disorder that can result from an abnormality associated with these factors, particularly lipid and glucose metabolism. Diabetes can manifest at any time during a person's lifetime. Another example of a disorder that relates to these factors is obesity. Obesity is characterized by excessive bodily adipose tissue (fat) and is a prevalent health issue. Many health complications can develop as a result of an adipose disorder such as obesity, and weight loss is often a difficult challenge for those suffering from such a condition. Additionally, once weight has been lost, maintaining a healthy weight is often difficult for those susceptible to an adipose disorder. The biochemical components of an individual's lipid metabolic processes affect lipid metabolism in the individual, and the relative degrees of expression and activity of those components among individuals can account for differences in lipid metabolism that are not clearly attributable to any disease or disorder. Thus, the ability to quantitatively characterize differences in the components of lipid metabolic pathways among individuals would permit individualization of treatment, diet, nutritional supplementation, and the like.

Adipose tissue is comprised primarily of adipocytes (fat cells). Adipocytes are hormone sensitive, lipid-storing cells that are located throughout the body. Dietary fats are hydrolyzed in the digestive tract to yield primarily fatty acids and monoglycerides. Digestive fatty acids and monoglycerides enter the lymph, are converted to triglycerides and other lipids and lipoproteins. Those products are transported, by way of the bloodstream, to adipose tissue and the liver in the form of lipid-containing particles including chylomicrons and high- and low-density lipoprotein particles. In adipose tissues, lipid are converted to fatty acids, transported across adipose cell membranes, and re-converted within adipose cells to triglycerides. Adipocytes serve as energy reservoirs for the body, and store lipids primarily in the form of triacylglycerides. When the body requires energy from stored lipids, hormones are generated that stimulate an enzyme known as hormone-sensitive lipase. Hormone-sensitive lipase breaks down triacylglycerides into free fatty acids and glycerol. The free fatty acids and glycerol are released from the adipocyte and transported to peripheral sites to provide energy to cells at those sites.

Susceptibility to developing an adipose disorder, such as obesity, has been linked to genetic factors. Some of these genetic factors are involved in regulating uptake, release, and metabolism of lipids. For example, the protein product of the obesity (ob) gene in mice (designated the LEP gene in humans) is a protein hormone designated leptin. Leptin is involved in regulation of adiposity. Leptin is synthesized primarily by adipocytes. Leptin level in the bloodstream is regulated by factors such as insulin and is roughly proportional to fat mass in humans. Circulating leptin level increases following feeding and decreases under starvation conditions. Once produced, leptin acts as a signal to the brain. In the brain, leptin binds with a leptin receptor protein (such as the protein encoded by the gene designated LEPR, which is sometimes referred to as OB-R and which is known to occur in highly homologous alternate forms). Binding of leptin with LEPR protein elicits a signaling cascade that leads to hunger reduction and lipid metabolism. The LEPR signaling pathway (a leptin signaling pathway in the nervous system) involves other gene products, for example, neuropeptide Y, melanocyte stimulating hormone, proopiomelanocortin, agouti gene-related protein (AGRP), and melanocortin-4 receptor. Therefore, leptin and LEPR, in conjunction with these other gene products, contributes to regulation of adiposity.

Most, if not all, human genes occur in a variety of forms which differ in at least minor ways. Heterogeneity in human genes is believed to have arisen, in part, from minor, non-fatal mutations that have occurred in the genome over time. In some instances, differences between alternative forms of a gene are manifested as differences in the amino acid sequence of a protein encoded by the gene. Some amino acid sequence differences can alter the reactivity, substrate specificity, or inter-protein binding specificity of the protein. Differences between alternative forms of a gene can also affect the degree to which (if at all) the gene is expressed. However, many heterogeneities that occur in human genes appear not to be correlated with any particular phenotype. Known heterogeneities include, for example, single nucleotide polymorphisms (i.e., alternative forms of a gene having a difference at a single nucleotide residue). Other known polymorphic forms include those in which the sequence of larger (e.g., 2-1000 residues) portions of a gene exhibits multiple sequence differences and those which differ by the presence or absence of portion of a gene.

Numerous disorders and physiological states have been correlated with occurrence of one or more alternative forms of a gene in the genome of a human who exhibits the disorder or physiological state. For example, Kimura et al. (2000, Am. J. Opthalmol. 130:769-773) discloses an association between occurrence of a SNP of the manganese superoxide dismutase gene and a form of macular degeneration. As another example, Mammes et al. (2001, Eur. J. Clin. Invest. 31(5):398-404) reports a relationship between LEPR gene polymorphisms and common obesity phenotypes. Although associations between individual disorders and individual genetic polymorphisms are known, a need remains for a method of assessing the overall state of adipose regulation and lipid metabolism in a human. Furthermore, a need exists for a method of correlating the state of an individual's lipid metabolic components with the individual's genomic content. The invention satisfies these needs.

### BRIEF SUMMARY OF THE INVENTION

The invention relates to a method of assessing relative susceptibility of a human to abnormal lipid metabolism. The invention also relates to correlating an individual's genetic composition with one or more of lipid metabolic state, susceptibility to hunger, susceptibility to lipid- or fat-induced satiety, susceptibility to weight gain, and resistance to weight gain in the individual. Each of these methods comprises assessing occurrence in the human's genome of one or more polymorphisms (e.g., single nucleotide polymorphisms; SNPs) in at least one gene (and preferably two genes) selected from the group consisting of
a) a gene which encodes leptin (specifically, the LEP gene); and,
b) a gene which encodes a leptin receptor (specifically, the LEPR gene).

Occurrence of any of the polymorphisms is an indication that the human is more susceptible to abnormal lipid metabolism than a human whose genome does not comprise the polymorphism. Abnormal lipid metabolism can be manifested, for example, as occurrence in the human of a readily-detectable disorder (e.g., obesity or diabetes) or as occurrence in the human of an enhanced propensity to gain weight, to resist gaining weight, to overeat, or to reduce caloric intake below maintenance levels. Furthermore, occurrence of a plurality of the polymorphisms is an indication that the human is even more susceptible to abnormal lipid metabolism than a human whose genome does not comprise the polymorphisms. Preferably, the genes are selected from the group consisting of a) and b). In one embodiment, the method comprises assessing occurrence in the human's genome of at least two of the aforementioned polymorphisms in a gene which encodes leptin (e.g., the LEPR gene).

The method by which occurrence of an individual disorder-associated polymorphism is assessed is not critical. For example, occurrence of the polymorphisms can be assessed using a method that includes contacting a nucleic acid derived from the human's genome with a first oligonucleotide. The first oligonucleotide can be one that anneals with higher stringency with the disorder-associated polymorphism than with a corresponding non-disorder-associated polymorphism. Annealing of the first oligonucleotide and the nucleic acid can be assessed, and such annealing is an indication that the human's genome comprises the disorder-associated polymorphism. Use of an oligonucleotide has the advantage that the oligonucleotide can be attached to a support using routine methods, and that a plurality of oligonucleotides can be attached to the same support, to allow simultaneous detection of multiple polymorphisms. If a second oligonucleotide which anneals with higher stringency with a non-disorder-associated polymorphism than with a corresponding disorder-associated polymorphism is used, then the allelic content (i.e., heterozygous or homozygous for one or the other polymorphic form) of the human's genome can be determined. Detection of polymorphic sequences can be simplified by using labeled oligonucleotides, such as molecular beacon oligonucleotides.

Once the content of the human's genome for disorder-associated polymorphisms has been assessed, assessment of susceptibility to abnormal lipid metabolism can further comprise calculating a susceptibility score for the human. A susceptibility score can be calculated by summing, for each of the disorder-associated polymorphisms that occurs in the human's genome, the product of a constant and a correlation factor. The correlation factor can, for example, be a factor that represents the fraction of humans heterozygous for the disorder-associated polymorphism who exhibit the corresponding disorder or a factor that represents the fraction of humans homozygous for the disorder-associated polymorphism who exhibit the corresponding disorder. The constant can be the same for each polymorphism, or it can be selected based on the known or surmised relevance of the corresponding gene with respect to lipid metabolism. The susceptibility score represents the relative susceptibility of the human to abnormal lipid metabolism.

In another aspect, the invention relates to a method of selecting a dose of a composition which alters leptin-modulated lipid metabolism (e.g., a composition comprising a compound that modulates leptin release, modulates binding of leptin to LEPR, or modulates the response induced upon binding of LEPR and leptin). Thus, this method can be used to identify compositions for administration to a human who exhibits, or is at risk for developing, abnormal lipid metabolism. This method comprises assessing occurrence in the human's genome of disorder-associated polymorphisms in at least one of the genes selected from the group consisting of a) and b) as indicated above. After assessing occurrence of the polymorphisms, a dose of the composition is selected. Occurrence of any of the polymorphisms is an indication that a greater dose of the composition should be administered to the human in whom the disorder-associated polymorphism occurs than to a human in whom the disorder-associated polymorphism does not occur.

The invention also relates to a kit for assessing relative susceptibility of a human to abnormal lipid metabolism. The kit comprises reagents for assessing occurrence in the human's genome of disorder-associated polymorphisms in at least one gene selected from the group consisting of a) and b) as indicated above. Examples of suitable reagents include oligonucleotides (e.g., molecular beacon oligonucleotides) that anneal with higher stringency with the disorder-associated polymorphisms than with corresponding non-disorder-associated polymorphisms and oligonucleotide primers that are complementary to the region adjacent a characteristic residue of the disorder-associated polymorphism. These primers are useful for amplifying at least the characteristic residue, thereby facilitating its detection. The kit can further comprise an instructional material which includes a numerical value representing the product of a constant and a correlation factor for some or all of the disorder-associated polymorphisms.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. The invention is not limited to the precise arrangements and instrumentalities shown.

FIGS. 1A and 1B are images which depict examples of results that can be obtained by analyzing occurrence of polymorphisms in several genes. The results shown in FIG. 1A are derived from a hypothetical first human, and those shown in FIG. 1B are derived from a hypothetical second human. Circles represent different polymorphisms of the gene indicated to the left of the row of circles. Filled circles indicate the presence of the polymorphism. Non-filled circles indicate the absence of the polymorphism. Numbers below each circle represent a correlation factor for the polymorphism and a disease or disorder.

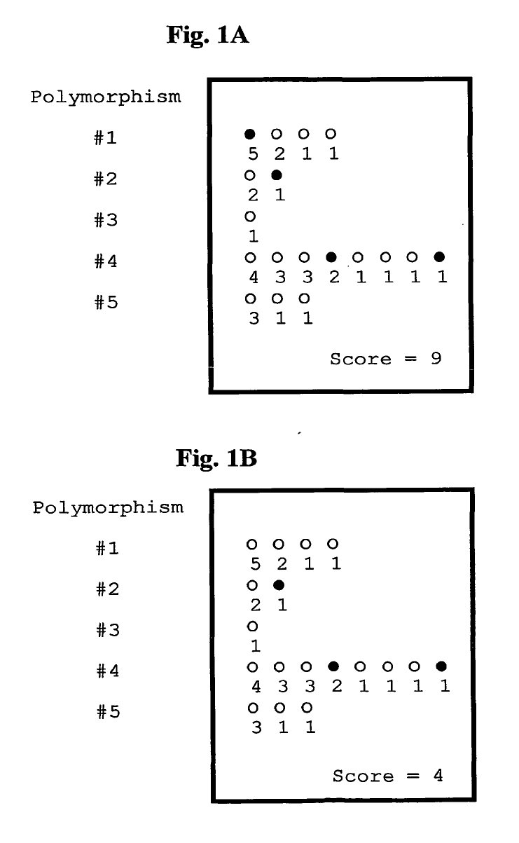

### DETAILED DESCRIPTION OF THE INVENTION

The invention relates to kits and methods for assessing the relative susceptibility of a human to abnormal lipid metabolism by assessing occurrence in the human's genome of genetic polymorphisms that are associated with disorders. Crudely simplified, the methods involve determining whether one or more polymorphisms that have been associated (by the inventors or by others) with a disorder (e.g., a disease or pathological state) in humans occur in a gene encoding a product associated with leptin-modulated lipid metabolism in the genome of the human being tested. In some embodiments, the number of polymorphisms that occur in the human's genome are summed to yield a value; the higher the value is, the greater the susceptibility of the human to abnormal lipid metabolism is assessed to be. In other embodiments, a weighting factor is assigned to each polymorphism tested, and the weighting factors of polymorphisms that occur in the human's genome are summed to yield a value that represents relative susceptibility to abnormal lipid metabolism. The weighting factor can, for example, represent the product of a constant assigned to the gene in which the corresponding polymorphism occurs and a correlation factor that describes how informative an occurrence of the polymorphism is for occurrence of the disorder with which it is associated. The invention includes a variety of alternative methods and kits for performing the methods, as described in greater detail herein.

### DEFINITIONS

As used in this disclosure, the following terms have the meanings associated with them in this section.

A “polymorphism” in a gene is one of the alternative forms of a portion of the gene that are known to occur in the human population. For example, many genes are known to exhibit single nucleotide polymorphic forms, whereby the identity of a single nucleotide residue of the gene differs among the forms. Each of the polymorphic forms represents a single polymorphism, as the term is used herein. Other known polymorphic forms include alternative forms in which multiple consecutive or closely-spaced, non-consecutive nucleotide residues vary in sequence, forms which differ by the presence or absence of a single nucleotide residue or a small number of nucleotide residues, and forms which exhibit different MRNA splicing patterns.

A “single nucleotide polymorphism” (“SNP”) is one of the alternative forms of a portion of a gene that vary only in the identity of a single nucleotide residue in that portion.

A “disorder-associated” polymorphism is an alternative form of a portion of a gene, wherein occurrence of the alternative form in the genome of a human has been correlated with exhibition by the human of a disease or a pathological state.

A “non-disorder-associated” polymorphism is an alternative form of a portion of a gene for which no significant correlation has been made between occurrence of the alternative form in the genome and a disease or a pathological state. Non-disorder-associated polymorphisms are sometimes designated “neutral” polymorphisms in the art.

A disorder-associated polymorphism and a non-disease-associated polymorphism “correspond” with one another if the two polymorphisms are two alternative forms of the same portion of the gene. By way of example, if the identity of residue 100 of a gene is adenine in a disorder-associated polymorphism of the gene and cytosine in a non-disorder-associated polymorphism of the gene, then the two polymorphisms correspond with one another. It is understood that there may be three or more corresponding polymorphisms when there are more than two alternative forms of the same portion of the gene.

A “characteristic residue” of a polymorphism is a nucleotide residue, the identity of which is known to vary among the alternative forms corresponding to the polymorphism.

An “adipose disorder” is a condition or pathological state associated with abnormal lipid metabolism.

An individual exhibits “abnormal lipid metabolism” if the individual exhibits a condition or pathological state wherein the degree or rapidity of at least one process selected from the group consisting of lipid uptake by adipocytes, lipid release from adipocytes, leptin-modulated hunger signaling, and leptin-modulated satiety signaling differs significantly (i.e., by at least 10%, 25%, 50%, 100%, 200%, or 500% or more) from the same process in a normal individual under the same nutritional conditions (e.g., an individual who has consumed an identical meal and waited an identical period of time prior to assessing the degree or rapidity of the process).

A “molecular beacon oligonucleotide” is a single-stranded oligonucleotide having a fluorescent label (e.g., rhodamine, FAM, TET, VIC, JOE, or HEX) attached to the 5′-end thereof and a fluorescence quencher (e.g., TAMRA or DABCYL) attached to the 3′-end thereof (or vice versa), as described (Kostrikis et al., 1998, Science 279:1228-1229).

Two molecular beacon oligonucleotides are “spectrally distinct” if they can be differentially detected using spectrophotometric or spectrofluorimetric methods. Examples of characteristics that can be used to differentiate spectrally distinct oligonucleotides include absorption or excitation wavelength, emission wavelength, and fluorescent lifetime.

An “instructional material” is a publication, recording, diagram, educational material, or any other medium of expression which can be used to communicate how to use a method or kit described herein or the assessment results of the inventors' methods. Alternatively, an instructional material may be advisory or educational. The instructional material can be used cooperatively with the kits or methods described herein.

A “control” is an actual or hypothetical individual whose genome does not comprise a single polymorphism which is assessed by the methods disclosed herein or an actual or hypothetical individual whose genome comprises a known quantity of polymorphisms assessed by the methods disclosed herein.

A “biological sample” is a biological sample including, for example and without limitation, blood, tissue, or urine, taken or collected from an individual for analysis, testing, storage, or assessment purposes.

An “effective dose” is the median dose that produces the desired therapeutic effect of the compound or composition administered. A minimum effective dose or a minimum therapeutically effective dose of a compound or composition can be estimated in a number of ways. For example, treatment doses can be compared to placebo doses to determine the lowest dosage at which the effect of the compound is statistically significant. (Filloon, T. G., et al., “Estimating the Minimum Therapeutically Effective Dose of a Compound via Regression Modelling and Percentile Estimation,” Stat. Med. 1995 May; 14(9-10): 925-32). As used herein, effective dose or “therapeutically effective dose” describes an amount of compound or composition which may be used to produce a favorable result according to the present invention.

An “intervention” is advising or employing consumption or use of a nutritional supplement or composition, advising or employing a nutritional supplement or composition, or advising or undergoing heightened medical monitoring.

A “nutrigenomic” composition is a customized nutritional supplement formulated to address an individual's supplementation needs based on the individual's predisposition to certain conditions as determined by assessment of the individual's genome. Nutrigenomic compositions are non-pharmaceutical; they are, however, compensatory and can be used to alleviate, inhibit, or prevent a disease state.

The “stringency” with which two polynucleotides anneal means the relative likelihood that the polynucleotides will anneal in a solution as the conditions of the solution become less favorable for annealing. Examples of stringent conditions are known in the art and can be found in available references (e.g., Current Protocols in Molecular Biology, John Wiley & Sons, N.Y., 1989, 6.3.1-6.3.6). Aqueous and non-aqueous annealing methods are described in that reference and either can be used. In general, a first pair of polynucleotides anneal with higher stringency than a second pair if the first pair is more likely to anneal (or remain annealed) as one or more of the salt concentration, temperature, and detergent concentration are increased.

With respect to a disorder, a “correlation factor” for a disorder-associated polymorphism is the fractions of humans who are heterozygous or homozygous for the polymorphism who exhibit the disorder. The correlation factor can, alternatively, be based solely on those who are heterozygous, solely on those who are homozygous, or on those who are either heterozygous or homozygous.

A “non-extendable” nucleotide residue is a nucleotide residue that is capable of being added to a polynucleotide by a polymerase (i.e., by extension of the polynucleotide in association with a complement thereof, catalyzed by the polymerase) and that, upon addition to the polynucleotide, renders the polynucleotide incapable of being further extended by the polymerase.

### DESCRIPTION

The invention relates to kits and methods for assessing the relative susceptibility of a human to abnormal lipid metabolism by assessing occurrence in the human's genome of genetic polymorphisms that are associated with one or more disorders.

It has been discovered that the degree to which a human is susceptible to abnormal lipid metabolism can be assessed by determining which polymorphic forms of certain genes are present in the human's genome. The relevant disorder-associated polymorphisms are those which occur in genes which encode products that are involved in leptin-mediated lipid metabolism and the associated intra- and inter-cellular signaling. Such products include not only leptin (i.e., the protein sometimes designated Ob, which is encoded by the OB gene in mice and the LEP gene in humans) and the leptin receptor (i.e., the protein encoded by a human gene alternatively designated LEPR and OB-R), but also can include products which are involved in leptin-mediated signaling, including proteins designated neuropeptide Y (encoded by the human NPY gene corresponding to GENBANK™ accession no. XM.sub.—004941), melanocyte stimulating hormone, proopiomelanocortin (encoded by the human POMC gene corresponding to GENBANK™ accession no. XM.sub.—2485), agouti gene-related protein (encoded by the human ART gene corresponding to GENBANK™ accession no. U88063), and melanocortin-4 receptor (encoded by the human MC4R gene corresponding to GENBANK™ accession no. XM.sub.—008716). The disorder with which a genetic polymorphism in a gene encoding one of these products is associated need not be a lipid metabolic disorder, or even a metabolic disorder of any type. Association of the polymorphism with any type of disease or disorder is an indication that that polymorphic form of the gene is aberrant and can contribute to a lipid metabolic disorder or to another form of abnormal lipid metabolism.

Polymorphisms associated with the leptin gene and the leptin receptor gene are known to be associated with various disorders (see, e.g., Mammes et al., 2001, Eur. J. Clin. Invest. 31:398-404 and Mammes et al., 2000, Ann. Hum. Genet. 64:391-394). Occurrence of disorder-associated polymorphisms in at least one (and preferably both) of these genes should be assessed in the methods described herein, given the importance of these genes. Similarly, the kits described herein preferably include reagents for detecting disorder-associated polymorphisms in at least one (and preferably both) of these genes. In addition, the significance of occurrence of disorder-associated polymorphisms in these genes can be applied by assigning a greater weighting factor to disorder-associated polymorphisms of these genes than to disorder-associated polymorphisms in other genes associated with leptin-mediated lipid metabolism.

Occurrence of disorder-associated polymorphisms in genes encoding products (e.g., the melanocortin 4 receptor encoded by the human MC4R gene corresponding to GENBANK™ accession no. SM.sub.—008716) that are involved in a leptin signaling pathway In a human are also an indication that the human is afflicted with, or is at risk for developing, abnormal lipid metabolism. Apart from leptin and the receptor encoded by the LEPR gene, such products include cell surface proteins and integral membrane proteins that are capable of binding leptin, transmembrane signaling proteins (e.g., G protein coupled receptors) that bind with extracellular leptin and effect a biochemical change intracellularly upon leptin binding, second message proteins which interact with such transmembrane signaling proteins, transcription factors which modulate gene expression in a manner that is responsive to extracellular leptin, transcription factors which modulate LEP gene expression in a manner that is responsive to an extracellular signal (e.g., in response to extracellular insulin level), and cell surface and transmembrane signaling proteins involved in transmitting an extracellular signal (e.g., insulin level) to an intracellular factor that modulates LEP gene expression. Many such products and their corresponding genes are known in the art.

Another group of genes for which occurrence therein of a disorder-associated polymorphism is indicative of an enhanced likelihood for, or risk of developing, abnormal lipid metabolism are genes which encode a protein for which the level of expression of the protein is associated (i.e., directly or conversely) with abnormal leptin-mediated lipid metabolism. For example, enhanced expression of the human gene (designated TNFA and corresponding to GENBANK™ accession no. X02910) encoding tumor necrosis factor alpha correlates with enhanced expression of leptin in patients afflicted with obesity and insulin resistance (Halle et al., 1998. Exerc. Immunol. Rev. 4:77-94). Occurrence of disorder-associated polymorphisms in such genes can provide direct or surrogate indication of the occurrence of, or risk for development of, abnormal lipid metabolism in a human.

It was not previously appreciated that detection in a human's genome of two or more disorder-associated polymorphisms in genes associated with leptin-mediated lipid metabolism is indicative that the human globally exhibits enhanced susceptibility to abnormal lipid metabolism. Previous studies are believed to have recognized only association between a single polymorphism in one of these genes and a particular disorder (e.g., obesity in the Mammes reference). The inventors believe that they are the first to describe methods and kits for assessing a human's global susceptibility to abnormal lipid metabolism. For example, a human may harbor gene polymorphs which render him susceptible to abnormal lipid metabolism that correlates to an inability to gain weight rather than correlating to obesity.

The polymorphisms in the foregoing genes which are assessed by the inventor's methods and are informative for assessing susceptibility to abnormal lipid metabolism include the following:

a polymorphism manifested as a change from a thymine to a cytosine at nucleotide residue +70 (in the first exon) of the leptin receptor (LEPR) gene;

a polymorphism manifested as a change from an Asp (A) to an Asp (G) at amino acid position 96 (in the fourth exon) of the leptin receptor (LEPR) gene;

a polymorphism manifested as a change from a Ser (T) to a Ser (C) at amino acid position 343 (in the ninth exon) of the leptin receptor (LEPR) gene; and

a polymorphism manifested as a change from a guanine residue to an adenine residue at nucleotide residue 2548 of the leptin (LEP) gene.

Each of the aforementioned polymorphisms has been associated with a disease state or other disorder. For example:

The polymorphism manifested as a change from a thymine to a cytosine at nucleotide residue +70 (in the first exon) of the leptin receptor (LEPR) gene has been associated with increased risk of obesity.

The polymorphism manifested as a change from an Asp (A) to an Asp (G) at amino acid position 96 (in the fourth exon) of the leptin receptor (LEPR) gene has been associated with obesity.

The polymorphism manifested as a change from a Ser (T) to a Ser (C) at amino acid position 343 (in the ninth exon) of the leptin receptor (LEPR) gene is more commonly found in overweight persons than persons of normal weight, indicating an association with obesity.

The polymorphism manifested as a change from a guanine residue to an adenine residue at nucleotide residue 2548 of the leptin (LEP) gene is related to significant increase in obesity risk.

Other disorder-associated polymorphisms that occur in genes associated with leptin-mediated lipid metabolism can be found in the art, and those polymorphisms can be used in the kits and methods described herein in the same manner as those polymorphisms explicitly disclosed herein.

Methods of Assessing Susceptibility to Abnormal Lipid Metabolism

The invention includes a method of assessing the relative susceptibility of a human to abnormal lipid metabolism. This susceptibility can be calculated relative to a hypothetical human whose genome does not contain a single disorder-associated polymorphism in a gene associated with lipid metabolism. Alternatively, susceptibility can be calculated relative to another human who may have one or more different disorder-associated polymorphisms than the human being assessed. In practice, the basis upon which raw susceptibility scores are calculated is immaterial, so long as the same basis is used for all humans whose scores are to be compared (i.e., so that the scores are relatable to one another).

The relative susceptibility of a human to abnormal lipid metabolism permits assessment of risks and benefits of a variety of compositions, conditions, and interventions. In one embodiment, susceptibility of a human to abnormal lipid metabolism can be used to determine whether the human would benefit by supplementing the human's ordinary nutritional intake with a composition that contains one or more nutritional supplements or neutriceutical components. Furthermore, relative susceptibility of the human to abnormal lipid metabolism can indicate an appropriate dose of such a composition. In another embodiment, suitability of a dietary regimen or intervention for a human can be determined by assessing the human's susceptibility to abnormal lipid metabolism.

Susceptibility of a human to abnormal lipid metabolism is assessed by assessing occurrence in the human's genome of a plurality of disorder-associated polymorphisms in one or more genes associated with abnormal lipid metabolism selected from the group consisting of a) and b). Occurrence of a disorder-associated polymorphism in one of these genes is an indication that the human has a greater susceptibility to abnormal lipid metabolism than a human in whose genome the polymorphism does not occur. Of course, occurrence of two, three, or more such polymorphisms in the human's genome indicates that the human exhibits even greater susceptibility to abnormal lipid metabolism.

Occurrence of every disorder-associated polymorphism in a gene related to lipid metabolism is not necessarily equally indicative of susceptibility to abnormal lipid metabolism. In order to account for differences in the significance of various disorder-associated polymorphisms, a weighting factor can be assigned to each polymorphism detected in the methods and kits described herein. As indicated above, two genes (leptin and leptin receptor) are known to have very significant roles in lipid metabolism in humans. All else being equal, disorder-associated polymorphisms that occur in one of these two genes are likely to be more significant than polymorphisms that occur in genes having less significant roles in lipid metabolism. Thus, a greater weighting factor can be assigned to these polymorphisms than to others. By way of example, the weighting factor assigned to these two polymorphisms can be 1.1 to 10 times (e.g., 5 times) greater than the weighting factor assigned to disorder-associated polymorphisms (having equal correlation with the corresponding disorder, as discussed below) in other genes. Preferably, the weighting factor assigned to polymorphisms in the leptin and LEPR genes is twice that assigned to disorder-associated polymorphisms in other genes.

Another factor which can influence the significance that is assigned to occurrence of a disorder-associated polymorphism in a human's genome is the degree to which the polymorphism is correlated with the corresponding disorder. Some disorders are highly correlated with occurrence of a genetic polymorphism, and other disorders exhibit lower correlation with a polymorphism. When a polymorphism is reported to be associated with a disorder (i.e., with a disease or pathological condition that need not be a lipid metabolism disorder, or even a metabolism disorder), a degree of correlation between the polymorphism and the disorder is often reported. One useful way of calculating a factor that describes correlation between a polymorphism and a disorder is to calculate an odds ratio that describes the likelihood that an individual in whose genome the disorder-associated polymorphism occurs will exhibit or develop the disorder. Because the kits and methods described herein can be used to detect whether the human is homozygous for the disease-associated polymorphism, odds ratios calculated for homozygous individuals can also be used, if they are available. Odds ratios can be calculated as described in the art.

For a disorder-associated polymorphism, the odds ratio can be calculated as follows. First, the odds of being afflicted with the disorder are calculated for a first population in whom the polymorphism occurs by dividing the number of afflicted individuals in the first population by the total number of individuals in the first population. Second, the odds of being afflicted with the disorder are calculated for a first population in whom the polymorphism does not occur by dividing the number of afflicted individuals in the second population by the total number of individuals in the second population. Third, the odds ratio is calculated by dividing the odds for the first population by the odds for the second population. If the odds ratio is greater than one, then this is an indication that occurrence of the polymorphism is associated with occurrence of the disorder. Furthermore, the magnitude of the odds ratio is an indication of the significance of the association.

An overall abnormal lipid metabolism susceptibility score for a human can be determined as follows. A significance score can be assigned to each disorder-associated polymorphism that is detected in the human's genome using a method or kit described herein. The significance score is a constant (e.g., 1.00), and is multiplied by any significance factor (e.g., 1-10, preferably 2 or 5, for the leptin {LEP or OB} and LEPR {or OB-R} genes) and by any correlation factor that is available. If information is available which describes the correlation between homozygosity for the polymorphism and the corresponding disorder, then that correlation factor should be used in place of the correlation factor for mere occurrence of the polymorphism, at least if the method or kit is used to rule out occurrence in the subject's genome of corresponding non-disorder-associated polymorphism(s). If significance and correlation factors are not available, then values of 1.00 should be assigned to each. An overall score is determined by summing the significance score for each disorder-associated polymorphism that is detected using the method or kit. This overall abnormal lipid metabolism susceptibility score can be compared with the values obtained from other subjects, or it can be compared with the value (i.e., zero) which would be expected to occur in a human whose genome does not include any disorder-associated polymorphism in a gene associated with abnormal lipid metabolism.

The method used to assess occurrence of any particular disorder-associated polymorphism (or non-disorder-associated polymorphism) is not critical. Numerous methods of detecting occurrence of a polymorphism are known in the art, and substantially any of those methods can be used in the kits and methods described herein. Naturally, the reagents included in the kit will vary depending on the method to be used to detect the polymorphisms. Examples of some suitable polymorphism detection methods are provided below.

In one embodiment, a pair of oligonucleotide primers are used to amplify a portion of the gene that includes a polymorphic region. Detection of one or more of the polymorphisms that occur at the polymorphic region can be achieved by contacting the amplified portion with an oligonucleotide having a sequence such that it will anneal under stringent conditions with the amplified portion only if one polymorphism occurs at the portion, but will not anneal with the amplified portion if another polymorphism occurs at that portion. Various acceptable stringent conditions are known in the art, and can be modified by the skilled artisan as appropriate to any particular amplified portion/oligonucleotide pair. An example of stringent conditions is hybridization in 6.times. sodium chloride/sodium citrate (SSC) at about 45.degree. C., followed by one or more washes in 0.2.times.SSC, 0.1% (w/v) SDS at 65.degree. C.

In an alternative embodiment, one or more molecular beacon oligonucleotides are used to detect polymorphisms (disorder-associated, non-disorder-associated, or both) in a sample that contains a copy of the subject's genome, a fraction of the subject's genome, or amplification products generated from the subject's genome (e.g., amplified portions of genes associated with lipid metabolism in which polymorphisms are known to occur).

Molecular beacon probes are single-stranded oligonucleotides having a fluorescent label (e.g., rhodamine, FAM, TET, VIC, JOE, or HEX) attached to the 5′-end thereof and a fluorescence quencher (e.g., TAMRA or DABCYL) attached to the 3′-end thereof (or vice versa), as described (Kostris et al., 1998, Science 279:1228-1229). The sequence of each molecular beacon probe is selected to include two complementary hairpin regions, whereby the probe can self-anneal to form a hairpin structure. The 5′- and 3′-ends are brought into close association when the hairpin structure forms. The probe also comprises a targeting portion which is selected to be complementary to a target sequence (e.g., a single polymorphism of a gene associated with lipid metabolism). The targeting portion and at least one of the hairpin regions are located in close proximity to one another, meaning that the targeting portion either overlaps the hairpin region or flanks it, having no more than about 5 nucleotide residues therebetween.

If the hairpin regions of the molecular beacon probe anneal with one another, then the probe does not fluoresce, because the hairpin structure forms and the fluorescence quencher attached to one end of the probe quenches fluorescence of the label attached to the other end of the probe. If the targeting portion of the probe anneals with a region of a nucleic acid having the target sequence, then formation of the hairpin structure is inhibited, the fluorescence quencher is not brought into association with the fluorescent label, and the probe fluoresces. Multiple molecular beacon probes can be used in a single reaction mixture, and fluorescence attributable to the probes can be differentiated if the molecular beacon probes are spectrally distinct.

Thus, in this embodiment, one or more molecular beacon probes are used, each having targeting portion which is complementary to a target region (e.g., 20 to 40 nucleotide residues, more preferably 20 to 30 residues) of one polymorphism of a gene associated with lipid metabolism (e.g., one of the genes disclosed herein). If the polymorphism to be detected is a single nucleotide polymorphism (SNP), then the target region includes, and preferably is approximately centered around, the nucleotide residue at which the polymorphism occurs. More preferably, two such probes are used, one having a targeting region completely complementary to the target region of one polymorphism of the gene (e.g., one of two polymorphisms of an SNP), and the other having a targeting region completely complementary to the target region of a corresponding polymorphism of the gene (e.g., the other polymorphism of the SNP). Preferably, this pair of probes are spectrally distinct.

In yet another embodiment of how polymorphisms in a gene associated with lipid metabolism can be assessed, oligonucleotide primers which are complementary to a region adjacent a characteristic residue of the polymorphism are extended using a polymerase enzyme, and the identity of the nucleotide residue that is added to the primer in the position complementary to the characteristic residue is determined. The primer can be extended in the presence of non-extendable nucleotide residues in order to ensure that a limited number of nucleotide residues (or only one residue) are incorporated into the primer. Methods of this type are known in the art (e.g., the SNP-IT.®™ technology of Orchid Biocomputer, Inc.) and are described, for example in U.S. Pat. Nos. 6,013,431 and 6,004,744.

Kits for Assessing Lipid Metabolism

The invention includes a kit for assessing the relative susceptibility of a human to abnormal lipid metabolism. The kit contains reagents for performing one or more of the methods described herein. The reagents used in certain embodiments of the methods described herein are indicated above. Reagents useful for performing those methods using a variety of alternative sample preparation and polymorphism detection methods or chemistries are apparent to the skilled artisan.

Kits for detecting polymorphisms in individual genes are known in the art, and the kit of the invention can have similar components. However, a critical feature of the kit is that it includes reagents that permit its user to detect at least three disorder-associated polymorphisms in genes associated with leptin-mediated lipid metabolism, such as the genes described herein (and preferably in both of those genes). Preferably the kit includes reagents that permit detection of at least 4, 6, 8, 10, 15, 20, or 30 or more disorder-associated polymorphisms in such genes.

In one embodiment, the kit includes a plurality of oligonucleotides which anneal under stringent conditions with a disorder-associated polymorphism of one of the genes, but not with a non-disorder associated-polymorphism. Each of the oligonucleotides is preferably attached to a surface in order to facilitate handling of the oligonucleotide. The oligonucleotides can be linked with a plurality of surfaces (e.g., oligonucleotides for a particular polymorphism being attached to a particle discrete from a particle to which oligonucleotides for another polymorphism are attached), or they can be attached to discrete regions of a single surface (e.g., as in the GENECHIP™ device of Affymetrix, Inc.). Annealing between individual oligonucleotides and the polymorphism corresponding thereto can be detected using standard methods. The kit can also comprise oligonucleotides that are useful as molecular beacon probes or as extendable primers.

In one embodiment, the kit further comprises a DNA collection kit or apparatus, such as that described in co-pending U.S. patent application Ser. No. 09/302,623 (allowed). Advantageously, DNA collected using the kit or apparatus can be stored or archived, and subjected to additional testing as previously unknown polymorphisms are discovered in genes associated with lipid metabolism, or as the significance of previously unappreciated polymorphisms is realized.

It will be appreciated by those skilled in the art that changes can made to the embodiments described above without departing from the broad inventive concept thereof.

This invention is not limited to the particular embodiments disclosed, and includes modifications within the spirit and scope of the present invention as defined by the appended claims.

What is claimed is:

1. A method of assessing the susceptibility of a human to abnormal lipid metabolism, the method comprising assessing occurrence in the human's genome of a plurality of polymorphisms in one or more genes selected from the group consisting of:
a) a gene which encodes leptin (LEP); and
b) a gene which encodes a leptin receptor (LEPR),
wherein the polymorphism in the in the LEP gene is manifested as a change from a guanine residue to an adenine residue at nucleotide residue 2548 and the polymorphisms in the LEPR gene consist of:
i) a polymorphism manifested as a change from a thymine to a cytosine at nucleotide residue +70 (in the first exon) of the LEPR gene;
ii) a polymorphism manifested as a change from an Asp (A) to an Asp (G) at amino acid position 96 (in the fourth exon) of the LEPR gene; and
iii) a polymorphism manifested as a change from a Ser (T) to a Ser (C) at amino acid position 343 (in the ninth exon) of the LEPR gene,
whereby occurrence of any of the polymorphisms is an indication that the human exhibits susceptibility to abnormal lipid metabolism, and whereby occurrence of a plurality of the polymorphisms is an indication that the human exhibits susceptibility to abnormal lipid metabolism.

2. A method of assessing the susceptibility of a human to abnormal lipid metabolism, the method comprising assessing occurrence in the human's genome of a plurality of polymorphisms in two genes, the genes consisting of a gene which encodes leptin (LEP) and a gene which encodes a leptin receptor (LEPR),
wherein the polymorphism in the LEP gene is manifested as a change from a guanine residue to an adenine residue at nucleotide residue 2548 and the polymorphisms in the LEPR gene consist of:
a) a polymorphism manifested as a change from a thymine to a cytosine at nucleotide residue +70 (in the first exon) of the LEPR gene;
b) a polymorphism manifested as a change from an Asp (A) to an Asp (G) at amino acid position 96 (in the fourth exon) of the LEPR gene; and
c) a polymorphism manifested as a change from a Ser (T) to a Ser (C) at amino acid position 343 (in the ninth exon) of the LEPR gene,
whereby occurrence of one or more of the polymorphisms is an indication that the human is susceptible to abnormal lipid metabolism.

3. The method of claim 2, the method further comprising advising an individual whose genome comprises one or more of the polymorphisms to employ an intervention.

4. A method of assessing the advisability that an individual should employ an intervention, the method comprising assessing occurrence in the human's genome of a plurality of polymorphisms consisting of:
a) a polymorphism in the LEP gene is manifested as a change from a guanine residue to an adenine residue at nucleotide residue 2548;
b) a polymorphism in the LEPR gene manifested as a change from a thymine to a cytosine at nucleotide residue +70 (in the first exon) of the LEPR gene;
c) a polymorphism in the LEPR gene manifested as a change from an Asp (A) to an Asp (G) at amino acid position 96 (in the fourth exon); and
d) a polymorphism manifested as a change from a Ser (T) to a Ser (C) at amino acid position 343 (in the ninth exon) of the LEPR gene,
whereby occurrence of one or more of the polymorphisms is an indication that the human is susceptible to abnormal lipid metabolism and should employ an intervention.
